(19) United States
(12) Patent Application Publication
(10) Pub. No.: US 2023/0375503 A1
(43) Pub. Date: Nov. 23, 2023

(54) GAS ANALYSIS DEVICE AND GAS ANALYSIS METHOD

(71) Applicant: HORIBA, LTD., Kyoto-shi, Kyoto (KR)

(72) Inventors: Ryota OCHIAI, Kyoto-shi, Kyoto (JP); Tomoshi YOSHIMURA, Kyoto-shi, Kyoto (JP); Ma Camille Corrales LACDAN, Kyoto-shi, Kyoto (JP)

(73) Assignee: HORIBA, LTD., Kyoto-shi, Kyoto (JP)

(21) Appl. No.: 18/030,877

(22) PCT Filed: Oct. 6, 2021
(86) PCT No.: PCT/JP2021/036943

§ 371 Date: Apr. 7, 2023

(30) Foreign Application Priority Data
Oct. 9, 2020 (JP) 2020-170943

(51) Int. Cl. G01N 27/626; G01N 1/22

(52) U.S. Cl. CPC G01N 27/626; G01N 1/2252; G01N 2001/2255

(57) ABSTRACT

A gas analysis device that comprises a first flow channel through which a sample gas flows, a first analyzer that is arranged in the first flow channel and that performs wet-measurement of a total hydrocarbon concentration in the sample gas, a second flow channel through which the sample gas flows, a non-methane cutter that is arranged in the second flow channel and that removes a hydrocarbon component other than methane in the sample gas, a second analyzer that is arranged downstream of the non-methane cutter in the second flow channel and that performs dry-measurement of a concentration of methane in the sample gas, and a calculation section that calculates a concentration of the hydrocarbon component other than methane in the sample gas using the total hydrocarbon concentration obtained by the first analyzer and a corrected concentration of methane, which is a moisture corrected methane concentration, obtained by the second analyzer.

### FIELD OF THE ART

This invention relates to a gas analysis device and a gas analysis method.

### BACKGROUND ART

Conventionally conceived is a gas analysis device that obtains a concentration of non-methane hydrocarbon (NMHC: Non-Methane Hydro-Carbon), which is obtained by removing methane (CH₄) from total hydrocarbon (THC: Total Hydro-Carbon) contained in an exhaust gas.

This gas analysis device comprises a detector (FID: Flame Ionization Detector) using a hydrogen flame ionization method, measures a concentration of THC in an exhaust gas and measures a concentration of methane contained in the exhaust gas that passes a non-methane cutter (NMC: Non-Methane Cutter) that removes a component of hydrogen other than methane in the exhaust gas. This gas analysis device then calculates the concentration of hydrocarbon component (NMHC) other than methane based on a difference in concentration obtained by these detectors. Normally, the gas analysis device measures the THC concentration or the methane concentration by, so called, wet-measurement after heating the exhaust gas or a flow channel without dehumidification to prevent the HC component from adsorbing to the flow channel or to prevent melting loss due to water droplet remaining in the flow channel. (Patent document 1).

The NMC used to measure the methane concentration is adjusted to a high temperature of 300° C. or higher to remove hydrocarbon component other than methane by oxidizing the hydrocarbon component other than methane through catalytic effect. However, since also methane is partially oxidized in the NMC at such high temperatures, a certain amount of water is added to the NMC to adjust the amount of methane oxidation. However, when measuring the methane concentration under a condition wherein the moisture concentration in the exhaust gas fluctuates greatly, the fluctuation in the moisture concentration leads to a fluctuation in a methane oxidized amount in the NMC, which significantly affects the measured value of the methane concentration. Therefore, it is preferable to measure the methane concentration using so-called dry-measurement by arranging a device such as a dehumidifier that keeps the moisture concentration in the exhaust gas constant in the upstream side of the NMC.

However, from a viewpoint of preventing dissolution loss of the HC component, it is preferable to measure the THC concentration by performing wet-measurement. Conventionally, since measurement conditions differ each other, a method to accurately calculate the NMHC concentration using the THC concentration measured by the wet-measurement and the methane concentration measured by the dry-measurement has not been established. The same can be said also in case of measuring the concentration of hydrocarbon other than methane and ethane (NMNEHC: Non-Methane Non-Ethane Hydro-Carbon) using non-methane/non-ethane cutter (NMNEC: Non-Methane Non-Ethane Cutter) that removes the component of hydrocarbon other than methane and ethane in the exhaust gas.

### PRIOR ART DOCUMENTS

### Patent Documents

Patent document 1: Japanese unexamined Patent Application Publication No. 2002-350304

### DISCLOSURE OF THE INVENTION

### Problems to be Solved by the Invention

The present claimed invention has been made to solve the above-mentioned problems, and a main object of this invention is to provide a gas analysis device that accurately calculates a concentration of NMHC and a concentration of NMNEHC in a sample gas.

### Means to Solve the Problems

More specifically, the gas analysis device of this invention is characterized by comprising a first flow channel through which a sample gas flows, a first analyzer that is arranged in the first flow channel and that performs wet-measurement of a total hydrocarbon concentration in the sample gas, a second flow channel through which the sample gas flows, a non-methane cutter that is arranged in the second tIow channel and that removes a hydrocarbon component other than methane in the sample gas, a second analyzer that is arranged downstream of the non-methane cutter in the second flow channel and that performs dry-measurement of a concentration of methane in the sample gas, and a calculation section that calculates the concentration of the hydrocarbon component other than methane in the sample gas using the total hydrocarbon concentration obtained by the first analyzer and a corrected methane concentration, which is a moisture-corrected methane concentration, measured by the second analyzer.

In accordance with this arrangement, it is possible to reduce fluctuations in an oxidized amount of methane in the non-methane cutter by performing dry-measurement of the methane concentration by arranging, for example, a dehumidifier that maintains a constant moisture concentration in the sample gas upstream of the non-methane cutter. It is possible to approach the measurement condition of the wet-measured THC concentration value by moisture-correcting the dry-measured methane concentration value and converting the moisture-corrected methane concentration value to a corrected methane concentration value that assumes, for example, wet-measurement. As a result of this, the NMHC concentration can be calculated with high accuracy while the THC concentration is wet-measured and the methane concentration is dry-measured. The NMHC includes, for example, hydrocarbon with a higher carbon number than methane and substituted functional groups, concretely alcohols, ethers, carboxylic acids, aldehydes, benzene, and esters.

As a concrete configuration of the gas analysis device, it is preferable that a heating section that applies heat to the sample gas is arranged in the first flow channel, and a moisture concentration adjustment section that adjusts a moisture concentration in the sample gas is arranged upstream of the non-methane cutter in the second flow channel. The heating section is preferably to apply heat to the sample gas at a temperature above the dew point temperature.

In addition, as a concrete configuration of the moisture concentration adjustment section represented is a dehumidifier that reduces the moisture concentration in the sample gas.

It is preferable that the corrected methane concentration is a methane concentration that considers the moisture concentration removed by the dehumidifier, and concretely, the corrected methane concentration is the methane concentration of the second analyzer multiplied by a predetermined moisture concentration correction coefficient.

In case that the second analyzer is calibrated by passing methane through the non-methane cutter, it can be conceived that the calculation section calculates the concentration of the hydrocarbon component other than methane in the sample gas according to the following expression (A).

$${x}_{NMHC} = \frac{K \cdot {x}_{THC ( NMC - FID )} \cdot {RF}_{{CH}_{4} ( THC - FID )} - {x}_{THC ( THC - FID )}}{\begin{matrix}{RF}_{{CH}_{4} ( THC - FID )} \cdot {RF}_{{C}_{2} {H}_{6} ( NMC - FID )} \cdot \\ {PF}_{{C}_{2} {H}_{6} ( NMC - FID )} - 1\end{matrix}} \qquad ( A )$$

x_{NMHC}: concentration of hydrocarbon component other than methane in sample gas
   x_{TCH (NMC-FID)}: concentration of methane measured by second analyzer
   RF_{CH4 (THC-FID)}: response coefficient of methane in first analyzer
   x_{THC (TCH-FID)}: total hydrocarbon concentration measured by first analyzer
   RF_{C2H6 (NMC-FID)}: response coefficient of ethane in second analyzer
   PF_{C2H6 (NMC-FID)}: transmittance of ethane in second analyzer
   K: moisture concentration correction coefficient

In addition, in case that the second analyzer is calibrated by using methane and bypassing the non-methane cutter, it can be conceived that the calculation section calculates the concentration of a hydrocarbon component other than methane in the sample gas using the following expression (B).

$${x}_{NMHC} = \frac{\begin{matrix}K \cdot {x}_{THC ( NMC - FID )} \cdot {RF}_{{CH}_{4} ( THC - FID )} - \\ {x}_{THC ( THC - FID )} \cdot P {F}_{{CH}_{4} ( NMC - FID )}\end{matrix}}{\begin{matrix}{RF}_{{CH}_{4} ( THC - FID )} \cdot {RF}_{{C}_{2} {H}_{6} ( NMC - FID )} \cdot \\ {PF}_{{C}_{2} {H}_{6} ( NMC - FID )} - P {F}_{{CH}_{4} ( NMC - FID )}\end{matrix}} \qquad ( B )$$

x_{NMHC}: concentration of hydrocarbon component other than methane in sample gas
   x_{TCH (NMC-FID)}: concentration of methane measured by second analyzer
   RF_{CH4 (THC-FID)}: response coefficient of methane in first analyzer
   x_{THC (TCH-FID)}: total hydrocarbon concentration measured by first analyzer
   PF_{CH4 (NMC-FID)}: transmittance of methane in second analyzer
   RP_{C2H6 (NMC-FID)}: response coefficient of ethane in second analyzer
   K: moisture concentration correction coefficient

In addition, in case that the second analyzer is calibrated by using propane and bypassing the non-methane cutter, it can be conceived that the calculation section calculates the concentration of the hydrocarbon component other than methane in the sample gas using the following expression (C).

$${x}_{NMHC} = \frac{K \cdot {x}_{THC ( NMC - FID )} - {x}_{THC ( THC - FID )} \cdot P {F}_{{CH}_{4} ( NMC - FID )}}{{PF}_{{C}_{2} {H}_{6} ( NMC - FID )} - {PF}_{{CH}_{4} ( NMC - FID )}} \qquad ( C )$$

x_{NHMC}: concentration of hydrocarbon component other than methane in the sample gas
   x_{TCH (NMC-FID)}: concentration of methane measured by second analyzer
   x_{THC (TCH-FID)}: total hydrocarbon concentration measured by first analyzer
   PF_{CH4 (NMC-FID)}: transmittance of methane in second analyzer
   PF_{C2H6 (NMC-FID)}: transmittance of ethane in second analyzer
   K: moisture concentration correction coefficient

It is preferable that the gas analysis device further comprises a third flow channel through which the sample gas flows, a non-methane/non-ethane cutter that is arranged in the third flow channel and that removes the hydrocarbon component other than methane and ethane in the sample gas and a third analyzer that is arranged downstream of the non-methane/non-ethane cutter in the third flow channel and that performs dry-measurement of the total methane/ethane concentration of methane and ethane in the sample gas, and the calculation section calculates the concentration of the hydrocarbon component other than methane and ethane in the sample gas using the total hydrocarbon concentration obtained by the first analyzer and the total corrected concentration of methane and ethane, which is the moisture-corrected total concentration of methane and ethane, obtained by the third analyzer.

In addition, in case that the third analyzer is calibrated by passing ethane through the non-methane/non-ethane cutter, it is preferable that the calculation section calculates the concentration of the hydrocarbon component other than methane in the sample gas according to the following expression (D).

$${x}_{NMNEHC} = \frac{{K}^{′} \cdot {x}_{THC ( NMNEC - FID )} - {x}_{THC ( THC - FID )}}{{PF}_{{C}_{3} {H}_{8} ( NMNEC - FID )} - 1} \qquad ( D )$$

x_{NMNHC}: concentration of hydrocarbon component other than methane and ethane in sample gas
   x_{TCH (NMNEC-FID)}: total concentration of methane and ethane measured by third analyzer
   x_{TCH (THC-FID)}: total hydrocarbon concentration measured by first analyzer
   PF_{C3H8 (NMNEC-FID)}: transmittance of propane in third analyzer
   K′: moisture concentration correction coefficient

A gas analysis device in accordance with this invention is characterized by comprising a first flow channel through which a sample gas flows, a first analyzer that is arranged in the first flow channel and that performs wet-measurement of a total hydrocarbon concentration in the sample gas, a second flow channel through which the sample gas flows, a non-methane/non-ethane cutter that is arranged in the second flow channel and that removes a hydrocarbon component other than methane and ethane in the sample gas, a second analyzer that is arranged downstream of the non-methane/non-ethane cutter in the second flow channel and that performs dry-measurement of a total concentration of methane and ethane in the sample gas, and a calculation section that calculates the concentration of the hydrocarbon component other than methane and ethane in the sample gas using the total hydrocarbon concentration obtained by the first analyzer and the total corrected concentration of methane and ethane, which is the moisture-corrected total concentration of methane and ethane, obtained by the second analyzer.

In accordance with this arrangement, it is possible to reduce fluctuations in an oxidized amount of methane and ethane in the non-methane and non-ethane cutter by performing dry-measurement of the methane concentration by arranging, for example, a dehumidifier that maintains a constant moisture concentration in the sample gas upstream of the non-methane and non-ethane cutter. It is possible to approach the measurement condition of the wet-measured THC concentration value by moisture-correcting the total concentration value of dry-measured methane and ethane and converting the moisture corrected methane and ethane concentration value to the total concentration value of corrected methane and ethane that assumes, for example, wet-measurement. As a result of this, the NMNEHC concentration can be calculated with high accuracy while the THC concentration is wet-measured and the total concentration of methane and ethane is dry-measured. The NMNEHC includes, for example, hydrocarbon with a higher carbon number than ethane and substituted functional groups, concretely alcohols, ethers, carboxylic acids, aldehydes, benzene, and esters.

In addition, a gas analysis device in accordance with this invention is characterized by comprising a first flow channel through which a sample gas flows, a first analyzer that is arranged in the first flow channel and that performs wet-measurement of a total hydrocarbon concentration in the sample gas, a second flow channel through which the sample gas flows, a hydrocarbon selective catalyst that is arranged in the second flow channel and that removes a predetermined hydrocarbon component in the sample gas, a second analyzer that is arranged downstream of the hydrocarbon selective catalyst in the second flow channel and that performs dry-measurement of the concentration of the hydrocarbon component in the sample gas, and a calculation section that calculates the concentration of a predetermined hydrocarbon component in the sample gas using the total hydrocarbon concentration obtained by the first analyzer and the corrected concentration of hydrocarbon component, which is the moisture-corrected concentration of the hydrocarbon component, obtained by the second analyzer.

In accordance with this gas analysis device, it is possible to accurately measure not only the concentration of hydrocarbon component other than methane and ethane (NMNEHC) in the sample gas but also the concentration of hydrocarbon component other than methane (NMHC) in the sample gas. In addition, it is possible to configure the non-methane/non-ethane cutter and non-methane cutter using a common hydrocarbon selective catalyst so that an arrangement of the flow channel can be simplified.

In addition, a gas analysis method in accordance with this invention is characterized by comprising steps of arranging a first analyzer that performs wet-measurement of a total hydrocarbon concentration in a sample gas in a first flow channel through which the sample gas flows, arranging a non-methane cutter that removes a hydrocarbon component other than methane in the sample gas in a second flow channel through which the sample gas flows, arranging a second analyzer that performs dry-measurement of a methane concentration in the sample gas downstream of the non-methane cutter in the second flow channel, and calculating the concentration of the hydrocarbon component other than methane in the sample gas using the total hydrocarbon concentration obtained by the first analyzer and a corrected methane concentration, which is a moisture corrected methane concentration, obtained by the second analyzer.

In accordance with the gas analysis method, it is possible to produce the same operation and effect as that of the gas analysis device of the above-mentioned invention.

### Effect of the Invention

In accordance with the gas analysis method of the present claimed invention, it is possible to provide a gas analysis device that can calculate a concentration of NMHC or a concentration of NMNEHC in a sample gas with high accuracy.

### BRIEF DESCRIPTION OF THE DRAWINGS

FIG. 1 A view schematically showing a configuration of the gas analysis device of this embodiment.

FIG. 2 A view schematically showing the configuration of the gas analysis device of other embodiments.

FIG. 3 A view schematically showing the configuration of the gas analysis device of another embodiment.

FIG. 4 A view schematically showing the configuration of the gas analysis device of further different embodiment.

### EXPLANATION OF CODES

100 . . . gas analysis device
L1 . . . first now channel
2 . . . first analyzer
L2 . . . second flow channel
3 . . . non-methane cutter (NMC)
4 . . . second analyzer
9 . . . moisture concentration adjustment section
11 . . . calculation section

### BEST MODES OF EMBODYING THE INVENTION

A gas analysis device 100 in accordance with one embodiment of this invention will be explained with reference to drawings.

### <Device Configuration>

The gas analysis device 100 of this embodiment analyzes hydrocarbon contained in an exhaust gas emitted from, for example, an internal combustion engine.

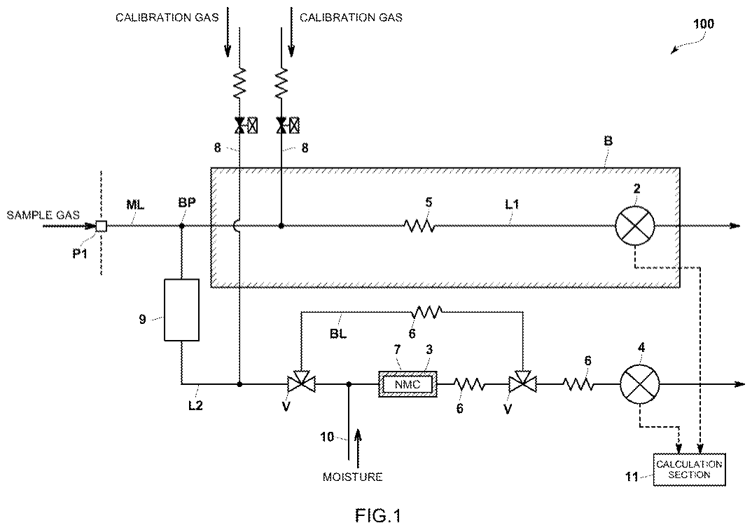

Concretely, as shown in FIG. 1, the gas analysis device 100 comprises a first flow channel L1 through which a sample gas as being an exhaust gas flows, a first analyzer 2 that is arranged in the first flow channel L1 and that measures a total hydrocarbon concentration (THC concentration) in the exhaust gas, a second flow channel L2 that is arranged separately from the first flow channel L1 and through which the exhaust gas flows, a non-methane cutter (NMC) 3 that is arranged in the second flow channel L2 and that removes a component of hydrocarbon (NMHC) other than methane in the exhaust gas and a second analyzer 4 that is arranged downstream of the non-methane cutter (NMC) 3 in the second low channel 12 and that measures the methane concentration in the exhaust gas.

The first flow channel L1 and the second flow channel L2 of this embodiment are branched at a predetermined branch point (BP) in a main flow channel (ML) that has an introducing port P1 through which the exhaust gas is introduced.

The first flow channel L1 and the second flow channel L2 are provided with a first flow rate adjustment mechanism 5 and a second flow rate adjustment mechanism 6 respectively to match a response timing of the first analyzer 2 with a response timing of the second analyzer 4.

The first flow rate adjustment mechanism 5 comprises, for example, a capillary arranged in an upstream side of the first analyzer 2 in the first flow channel L1, and the second flow rate adjustment mechanism 6 comprises, for example, a capillary arranged in an upstream side of the second analyzer 4 in the second flow channel L2.

The NMC 3 arranged in the second flow channel L2 is an oxidation catalyst and may use, for example, manganese dioxide, copper oxide, platinum or the like and may be any other metal that can be the oxidation catalyst. In addition, the NMC 3 is heated to 300° C. or higher. Concretely, the second flow channel L2 is provided with a heating section 7 for heating the NMC 3 to the above-mentioned temperature. By heating the NMC 3 to this temperature, it is possible for the NMC 3 to efficiently burn and remove the component of hydrocarbon other than methane and to pass methane efficiently (for example, 80% or more).

Furthermore, a bypass line (BL) that branches off from the upstream side of the NMC 3 and joins the downstream side thereof and a switching valve (V) such as a three-way valve that allows the exhaust gas to flow selectively to the NMC 3 or the bypass line (BL) are provided in this embodiment.

The first analyzer 2 and the second analyzer 4 are FID detectors that detect hydrocarbon in the exhaust gas by the hydrogen flame ionization (FID) method. A fuel gas (for example, H₂, or H₂/He mixture gas) and air for supporting combustion are supplied to the FID detectors from a gas suppling line, not shown in drawings. In addition, a calibration gas supplying line 8, which supplies a calibration gas for calibrating the first analyzer 2 and the second analyzer 4, is connected to the first flow channel L1 and the second flow channel L2 respectively.

The gas analysis device 100 is so configured that the first analyzer 2 performs wet-measurement of the total hydrocarbon concentration in the exhaust gas, and the second analyzer 4 performs dry-measurement of the methane concentration in the exhaust gas.

Concretely, a heating section (a heating block) (B) that applies heat to the flow channel and the first analyzer 2 up to a predetermined temperature (for example, 191° C.) is arranged in the first flow channel L1. The predetermined temperature is preferably over the dew point temperature in accordance with the pressure of the exhaust gas.

On the other hand, a moisture concentration adjustment section 9 that adjusts the moisture concentration in the exhaust gas is arranged upstream of the NMC 3 in the second flow channel L2. The moisture concentration adjustment section 9 is configured to change the temperature of the exhaust gas and to keep the moisture concentration constant, and to lower the moisture concentration contained in the sample gas up to a predetermined set concentration. The moisture concentration adjustment section 9 makes use of a dehumidifier that cools the exhaust gas introduced into the NMC 3 up to below the dew point temperature and dehumidifies the exhaust gas. The dehumidifier may be, for example, an electronic cooler that performs cooling by making use of, for example, the Peltier effect or a compressor type cooler using a compressor. A humidifier may be provided in an upstream side of the dehumidifier in the second flow channel L2 to raise the moisture concentration in the exhaust gas introduced into the dehumidifier above the saturated water vapor content in the dehumidifier.

A moisture supplying line 10 that supplies moisture to the second flow channel L2 is connected between the moisture concentration adjustment section 9 and the NMC 3 in the second flow channel L2. The moisture supplying line 10 is provided with a regulator, not shown in drawings, that is configured to control an amount of the moisture supplied to the second flow channel L2 to a predetermined amount. In this embodiment, the moisture supplying line 10 is configured to supply 1 to 2 vol % of the moisture to the second flow channel L2.

The gas analysis device 100 comprises a calculation section 11 that calculates the concentration of NMHC in the exhaust gas based on each of the concentrations obtained by the first analyzer 2 and the second analyzer 4.

The calculation section 11 of this embodiment calculates the concentration of NMHC in the exhaust gas using the THC concentration measured by the first analyzer 2 and a corrected methane concentration, which is a moisture corrected methane concentration, measured by the second analyzer 4.

Concretely, the corrected methane concentration is the methane concentration that takes into account the moisture concentration removed by the dehumidifier, and more concretely, the corrected methane concentration is calculated by multiplying the methane concentration measured by the second analyzer 4 by a predetermined moisture concentration correction coefficient (K).

The moisture concentration correction coefficient (K) converts the dry-measured methane concentration into a measured value in case of performing wet-measurement, and is expressed by the following expression (a) or an equivalent expression.

$$K = \frac{1 - {x}_{{H}_{2} O \cdot w}}{1 - {x}_{{H}_{2} O \cdot d}} \qquad ( a )$$

Where, x_{H2O·w}, is the moisture concentration in the exhaust gas before adjusting the moisture concentration in an upstream side of the moisture concentration adjustment section 9 in the second flow channel L2, and x_{H2O·d} is the moisture concentration in the exhaust gas after adjusting the moisture concentration in a downstream side of the moisture concentration adjustment section 9 in the second flow channel L2.

More concretely, the calculation section 11 calculates the NMHC concentration in accordance with a calibration method of the second analyzer 4 using the following expressions (1) through (3).
(A) In case of calibrating the second analyzer 4 using methane that passes through the NMC 3

$${x}_{NMHC} = \frac{K \cdot {x}_{THC ( NMC - FID )} \cdot {RF}_{{CH}_{4} ( THC - FID )} - {x}_{THC ( THC - FID )}}{\begin{matrix}{RF}_{{CH}_{4} ( THC - FID )} \cdot {RF}_{{C}_{2} {H}_{6} ( NMC - FID )} \cdot \\ {PF}_{{C}_{2} {H}_{6} ( NMC - FID )} - 1\end{matrix}} \qquad ( 1 )$$

(B) In case of calibrating the second analyzer 4 using methane that bypasses the NMC 3

$${x}_{NMHC} = \frac{\begin{matrix}K \cdot {x}_{THC ( NMC - FID )} \cdot {RF}_{{CH}_{4} ( THC - FID )} - \\ {x}_{THC ( THC - FID )} \cdot P {F}_{{CH}_{4} ( NMC - FID )}\end{matrix}}{\begin{matrix}{RF}_{{CH}_{4} ( THC - FID )} \cdot {RF}_{{C}_{2} {H}_{6} ( NMC - FID )} \cdot \\ {PF}_{{C}_{2} {H}_{6} ( NMC - FID )} - P {F}_{{CH}_{4} ( NMC - FID )}\end{matrix}} \qquad ( 2 )$$

(C) In case of calibrating the second analyzer 4 using propane that bypasses the NMC 3

$${x}_{NMHC} = \frac{K \cdot {x}_{THC ( NMC - FID )} - {x}_{THC ( THC - FID )} \cdot P {F}_{{CH}_{4} ( NMC - FID )}}{{PF}_{{C}_{2} {H}_{6} ( NMC - FID )} - {PF}_{{CH}_{4} ( NMC - FID )}} \qquad ( 3 )$$

Where,
x_{NMNHC}: concentration of hydrocarbon component other than methane in exhaust gas
x_{TCH (THC-FID)}: total hydrocarbon concentration obtained by first analyzer 2
x_{TCH (NMC-FID)}: total hydrocarbon concentration obtained by second analyzer 4
RF_{CH4 (THC-FID)}: response coefficient of methane in first analyzer 2
RF_{C2H6 (NMC-FID)}: response coefficient of ethane in second analyzer 4
PF_{CH4 (NMC-FID)}: transmittance of methane in second analyzer 4
PF_{C2H6 (NMC-FID)}: transmittance of ethane in second analyzer 4
K: moisture concentration correction coefficient

Derivation of the above-mentioned expression (1) through (3) will be explained below.

First, the THC concentration x_{THC [TCH-FID]} wet-measured by the first analyzer 2 is expressed by the following expression (4) using the response coefficient RF_{CH4 [THC-FID]} of methane in the first analyzer 2, the methane concentration x_{CH4} in the sample gas and the concentration x_{NMHC} of the hydrocarbon component other than methane in the sample gas.

x_{THC [TCH-FID]}=x_{CH4}×RF_{CH4 [THC-FID]}+x_{NMHC} (4)

The expression (4) assumes that the first analyzer 2 (THC-FID) is calibrated using a standard gas of propane C₃H₈. Therefore, RF_{CH4 [THC-FID]} is the response coefficient of methane in the first analyzer 2 which is propane-calibrated. The response coefficient is the sensitivity difference (ratio) of the target component to the calibration gas.

The methane concentration obtained by the second analyzer 4 (NMC-FID) has a different concentration calculation expression depending on the calibration method.

(A) In case of calibrating the second analyzer 4 using methane that passes through the NMC 3

In this case, the methane concentration dry-measured by the second analyzer 4 is expressed by the following expression (5).

x_{TCH [NMC-FID]}=[x_{CH4}+x_{NMHC}×RF_{C2H6 [NMC-FID]}×PF_{C2H6 [NMC-FID]}]/K (5)

(B) in case of calibrating the second analyzer 4 using methane that bypasses the NMC 3

In this case, the methane concentration dry-measured by the second analyzer 4 is expressed by the following expression (6).

x_{TCH [NMC-FID]}=[x_{CH4}×PF_{CH4 [NMC-FID]}+x_{NMHC}×RF_{C2H6 [NMC-FID]}×PF_{C2H6 [NMC-FID]}]/K (6)

(C) In case of calibrating the second analyzer 2 using propane that bypasses the NMC 3

In this case, the methane concentration dry-measured by the second analyzer 4 is expressed by the following expression (7).

x_{TCH [NMC-FID]}=[x_{CH4}×RF_{CH4 [NMC-FID]}×PF_{CH4 [NMC-FID]}+x_{NMHC}×RF_{C2H6 [NMC-FID]}×PF_{C2H6 [NMC-FID]}]/K (7)

It is possible to obtain the expressions (1) through (3) respectively as being calculation expressions of the NMHC concentration in accordance with the above-mentioned calibration method of the second analyzer 4 based on the above-mentioned expression (4) that expresses the THC concentration wet-measured by the first analyzer 2, and the above-mentioned expressions (5) through (7) that express the methane concentration dry-measured by the second analyzer 4.

<Effects of this Embodiment>

In accordance with the gas analysis device 100 of this embodiment, it is possible to make the dry-measured THC concentration value closer to the measurement conditions of the wet-measured THC concentration value by moisture-correcting the dry-measured methane concentration and by converting the moisture-corrected methane concentration to a corrected methane concentration value that assumes to be, for example, wet-measured. As a result of this, the NMHC concentration can be calculated with high accuracy while the THC concentration is wet-measured and the methane concentration is dry-measured.

### <Other Modified Embodiments>

The present claimed invention is not limited to the above-mentioned embodiments.

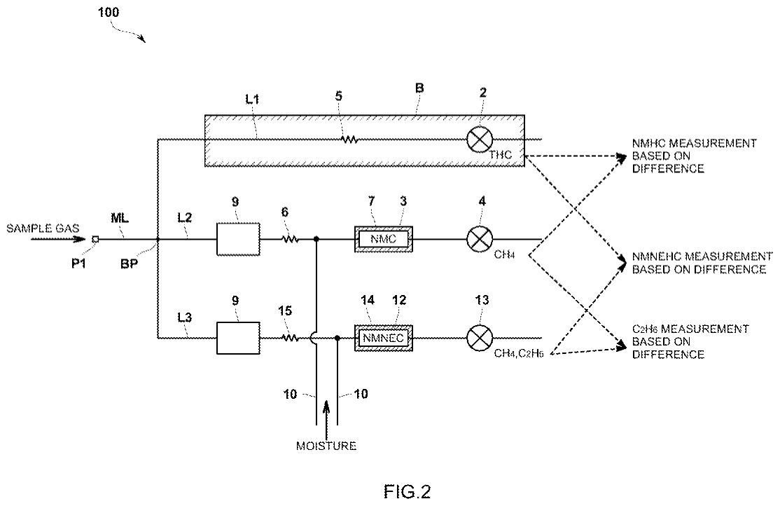

As shown in FIG. 2, the gas analysis device 100 may further comprise a third flow channel L3 through which the sample gas flows and where a non-methane/non-ethane cutter (NMNEC) 12, which removes hydrocarbon component other than methane and ethane in the sample gas, is arranged and a third analyzer 13 that performs dry-measurement of a total concentration of methane and ethane in the sample gas. In this case, a heating section 14 that applies heat to the NMNEC 12 to 200˜250° C. is arranged in the third flow channel L3. In addition, the moisture concentration adjustment section 9 (for example, dehumidifier, or the like) that adjusts the moisture concentration in the sample gas is arranged upstream of the NMNEC 12 in the third flow channel L3, and a water supplying line 10 is connected between the moisture concentration adjustment section 9 and the NMNEC 12 in the third flow channel L3.

In this case, the above-mentioned calculation section 11 (not shown in FIG. 2) can calculate the concentration of hydrocarbon component (NMNEHC) other than methane and ethane in the sample gas using the THC concentration of the first analyzer 2 and the corrected total concentration of methane and ethane, which is the moisture-corrected total concentration of methane and ethane of the third analyzer 13. This moisture correction is performed using the same method as that of the above-mentioned moisture correction of the methane concentration. In addition, similar to the above-mentioned calculation of the concentration of the hydrocarbon component (NMHC) other than methane, the calculation section 11 calculates the concentration of hydrocarbon component (NMNEHC) other than methane and ethane based on a predetermined calculation expression according to the calibration method of the third analyzer 13.

Concretely, the corrected total concentration of methane and ethane is the total concentration of methane and ethane that takes into account the moisture concentration removed by the dehumidifier, and more concretely, the corrected total concentration of methane and ethane is calculated by multiplying the total concentration of methane and ethane measured by the third analyzer 13 by a predetermined moisture concentration correction coefficient (K′).

The moisture concentration correction coefficient (K′) converts the dry-measured total concentration of methane and ethane to the measured value in case of performing wet-measurement, and is expressed by the following expression (b) or its equivalent expression.

$${K}^{′} = \frac{1 - {{x}^{′}}_{{H}_{2} O \cdot w}}{1 - {{x}^{′}}_{{H}_{2} O \cdot d}} \qquad ( b )$$

Where,

x′_{H2O·w} is a moisture concentration in the exhaust gas before adjusting a moisture concentration in an upstream side of the moisture concentration adjustment section 9 in the third flow channel L3, and x′_{H2O·d} is a moisture concentration in the exhaust gas in a downstream side of the moisture concentration adjustment section 9 in third flow channel L3.

Then, the calculation section 11 calculates the NMNEHC concentration according to the following expression (8).

$${x}_{NMNEHC} = \frac{{K}^{′} \cdot {x}_{THC ( NMNEC - FID )} - {x}_{THC ( THC - FID )}}{{PF}_{{C}_{3} {H}_{8} ( NMNEC - FID )} - 1} \qquad ( 8 )$$

Where:
x_{NMNEHC}: concentration of hydrocarbon component other than methane and ethane in exhaust gas
x_{THC (TCH-FID)}: total hydrocarbon concentration measured by first analyzer 2
x_{THC (NMNEC-FID)}: total concentration of methane and ethane measured by third analyzer 13
PF_{C3H8 (NMNEC-FID)}: transmittance of propane in third analyzer 13
K′: moisture concentration correction coefficient

The derivation of the above-mentioned expression (8) is explained below.

First, the THC concentration (x_{THC [TCH-FID]}) wet-measured by the first analyzer 2 becomes the following expression (9) using the response coefficient (RF_{CH4 [THC-FID]}) of methane in the first analyzer 2, the response coefficient (RF_{C2H6 [THC-FID]}) of ethane in the first analyzer 2, the methane concentration (x_{CH4}) in the sample gas, the ethane concentration (x_{C2H6}) in the sample gas, and the concentration (x_{NMNEHC}) of hydrocarbon component other than methane and ethane in the sample gas.

x_{THC [TCH-FID]}=x_{CH4}×RF_{CH4 [THC-FID]}+x_{C2H6}×RF_{C2H6 [THC-FID]}+x_{NMNEHC} (9)

This expression (9) presupposes that the first analyzer 2 (THC-FID) is calibrated using a standard gas of propane C₃H₈. Therefore, each of RF_{CH4 [THC-FID]} and RF_{C2H6 [THC-FID]} is the response coefficient of methane and the response coefficient of ethane respectively in the first analyzer 2 that is propane calibrated. The response coefficient is a sensitivity difference (ratio) between a target component and the calibration gas. In this embodiment, since the sensitivity difference of ethane to propane C₃H₈ can be regarded to be 1, the response coefficient RF_{C2H6 [THC-FID]} of ethane in the first analyzer 2 can be approximated as 1.

Next, in case that the third analyzer 13 (NMNEC-FID) is calibrated using ethane as the calibration gas that passes through the NMNEC 12, the total concentration of methane and ethane obtained by performing dry-measurement by the third analyzer 13 (NMNEC-FID) is expressed by the following expression (10).

x_{THC [NMNEC-FID]}=[x_{CH4}×RF_{CH4 [NMNEC-FID]}×PF_{CH4 [NMNEC-FID]}+x_{C2H6}+x_{NMNEHC}×RF_{C3H8 [NMNEC-FID]}×PF_{C3H8 [NMNEC-FID]}]/K′ (10)

Where,
RF_{CH4 [NMNEC-FID]}: response coefficient of methane in third analyzer 13
PF_{CH4 [NMNEC-FID]}: transmittance of methane in third analyzer 13
RF_{C3H8 [NMNEC-FID]}: response coefficient of propane in third analyzer 13
PF_{C3H8 [NMNEC-FID]}: transmittance of propane in third analyzer 13.

The NMNEX 12 is considered to transmit all methane in the sample gas because the NMNEC 12 is heated at 200˜250° C. Then it is possible to approximate PF_{CH4 [NMNEC-FID]} as 1. Furthermore, since the sensitivity difference between propane and ethane can be considered to be 1, it is possible to approximate the response coefficient RF_{C3H8 [NMNEC-FID]} of propane in the third analyzer 13 as 1.

Then, it is possible to obtain the expression (8), which is an expression for calculating the above-mentioned NMNEHC concentration, using the above-mentioned expression (9) expressing the THC concentration wet-measured by the first analyzer 2 and the above-mentioned expression (10) expressing the total concentration of methane and ethane dry-measured by the third analyzer 13.

In addition, it is also possible for the calculation section 11 to calculate the ethane concentration in the sample gas using the calibrated methane concentration measured by the second analyzer 4 and the calibrated total concentration of methane and ethane measured by the third analyzer 13.

Furthermore, a third flow rate adjustment mechanism 15 is arranged in the third flow channel L3 to synchronize the response timing of the first analyzer 2, the second analyzer 4 and the third analyzer 13. The third flow rate adjustment mechanism 15 consists of, for example, a capillary arranged in an upstream side of the third analyzer 13 in the third flow channel L3.

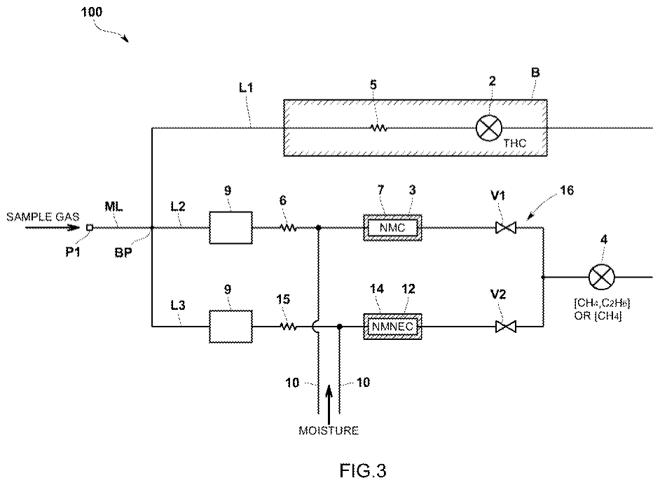

In addition, as shown in FIG. 3, the gas analysis device 100 may merge the downstream side of the NMC 3 in the second flow channel L2 and the downstream side of the NMNEC in the third flow channel L3 and introduce the sample gas into a common analyzer. In this case, a flow channel switch mechanism 16 comprising, for example, open/close valves V1, V2 is arranged to switch the flow channel through which the sample gas flows between the second flow channel L2 and the third flow channel L3. When switched to the second flow channel L2, the common analyzer performs dry-measurement of the methane concentration. When switched to the third flow channel L3, the common analyzer performs dry-measurement of the total concentration of methane and ethane. In accordance with this configuration, it is possible to reduce the number of the analyzer so that a cost of the gas analysis device 100 can be lowered.

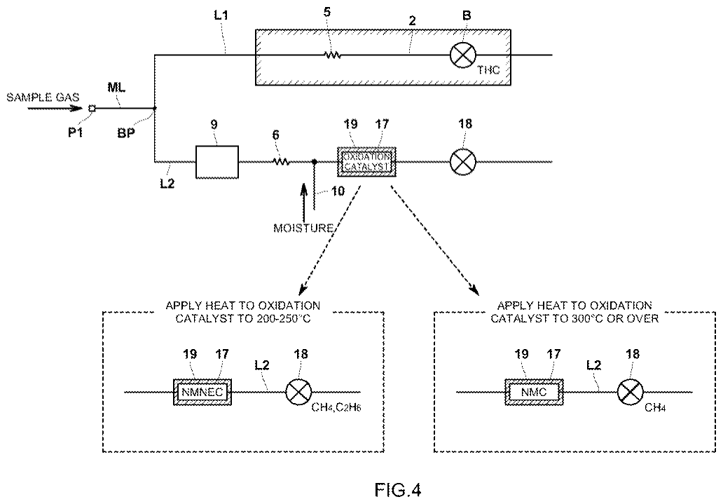

Moreover, as shown in FIG. 4, the gas analysis device 100 may comprise the first flow channel L1 through which the exhaust gas as the sample gas flows, the first analyzer 2 that performs wet-measurement of the total hydrocarbon concentration (THC concentration) in the exhaust gas, the second flow channel L2 that is arranged separately from the first flow channel L1 and through which the exhaust gas flows, the moisture concentration adjustment section 9 (for example, a dehumidifier) that adjusts the moisture concentration in the sample gas, a hydrocarbon selective catalyst 17 that removes a specified hydrocarbon component in the exhaust gas, an analyzer 18 that is arranged downstream of the hydrocarbon selective catalyst 17 in the second flow channel L2 and that performs dry-measurement of the concentration of hydrocarbon component in the exhaust gas and a temperature switching mechanism 19 that switches the temperature of the hydrocarbon selective catalyst 17. The analyzer 18 is an FID detector that detects hydrocarbon in the exhaust gas by the hydrogen flame ionization (FID) method. The hydrocarbon selective catalyst 17 is a catalyst that can select the hydrocarbon component to be removed according to the temperature, and the hydrocarbon selective catalyst 17 becomes a non-methane cutter or a non-methane/non-ethane cutter depending on the temperature. The hydrocarbon selective catalyst 17 may selectively remove not only methane, or methane and ethane, but also other hydrocarbon. Similar to the above-mentioned embodiment, the temperature switching mechanism 19 comprises a heating section whose temperature setting can be changed.

The temperature switching mechanism 19 applies heat to the hydrocarbon selective catalyst 17 to 200˜250° C. to make the hydrocarbon selective catalyst 17 a non-methane/non-ethane cutter that removes the component of hydrocarbon other than methane and ethane in the exhaust gas. With this arrangement, the analyzer 18 performs dry-measurement of the total concentration of methane and ethane in the exhaust gas. In this embodiment, the calculation section 11 of the gas analysis device 100 calculates the concentration of hydrocarbon component other than methane and ethane in the exhaust gas based on the THC concentration of the first analyzer 2 and the corrected total concentration of methane and ethane, which is the moisture-corrected total concentration of methane and ethane, of the analyzer 18.

In addition, the temperature switching mechanism 19 applies heat to the hydrocarbon selective catalyst 17 to 300° C. or higher to make the hydrocarbon selective catalyst 17 a non-methane cutter that removes hydrocarbon component other than methane in the exhaust gas. As a result of this, the analyzer 18 performs dry-measurement of the concentration of methane in the exhaust gas. In this embodiment, the calculation section 11 of the gas analysis device 100 calculates the concentration of hydrocarbon component other than methane in the exhaust gas based on the THC concentration of the first analyzer 2 and the corrected concentration of methane, which is the moisture-corrected concentration of methane, of the analyzer 18.

Switching the temperature by the temperature switching mechanism 19 may be configured to be set manually by a user or to be set automatically, for example, with a predetermined measurement sequence. In the above-mentioned explanation, the analyzer is assumed to be the FID detector, but any analyzer that can measure hydrocarbon component may be used.

In addition, various modifications and combinations of embodiments may be made without departing from the spirit of this invention.

### POSSIBLE APPLICATIONS IN INDUSTRY

In accordance with the present claimed invention, it is possible to provide a gas analysis device that can accurately calculate the concentration of NMHC and the concentration of NMNEHC in the sample gas.

1. A gas analysis device comprising
a first flow channel through which a sample gas flows,
a first analyzer that is arranged in the first flow channel and that performs wet-measurement of a total hydrocarbon concentration in the sample gas,
a second flow channel through which the sample gas flows,
a non-methane cutter that is arranged in the second flow channel and that removes a hydrocarbon component other than methane in the sample gas,
a second analyzer that is arranged downstream of the non-methane cutter in the second flow channel and that performs dry-measurement of a concentration of methane in the sample gas, and
a calculation section that calculates a concentration of the hydrocarbon component other than methane in the sample gas using the total hydrocarbon concentration obtained by the first analyzer and a corrected concentration of methane, which is a moisture-corrected methane concentration, obtained by the second analyzer.

2. The gas analysis device described in claim 1, wherein
a heating section that applies heat to the sample gas is arranged in the first flow channel, and
a moisture concentration adjustment section that adjusts a moisture concentration in the sample gas is arranged upstream of the non-methane cutter in the second flow channel.

3. The gas analysis device described in claim 2, wherein
the moisture concentration adjustment section is a dehumidifier that reduces the moisture concentration in the sample gas.

4. The gas analysis device described in claim 3, wherein
the corrected methane concentration is the methane concentration that considers the moisture concentration removed by the dehumidifier.

5. The gas analysis device described in claim 1, wherein
the corrected methane concentration is the methane concentration of the second analyzer multiplied by a predetermined moisture concentration correction coefficient.

6. The gas analysis device described in claim 1,
in case that the second analyzer is calibrated by passing methane through the non-methane cutter,
the calculation section calculates the concentration of the hydrocarbon component other than methane in the sample gas according to the following expression (A).
$${x}_{NMHC} = \frac{K \cdot {x}_{THC ( NMC - FID )} \cdot {RF}_{{CH}_{4} ( THC - FID )} - {x}_{THC ( THC - FID )}}{\begin{matrix}{RF}_{{CH}_{4} ( THC - FID )} \cdot {RF}_{{C}_{2} {H}_{6} ( NMC - FID )} \cdot \\ {PF}_{{C}_{2} {H}_{6} ( NMC - FID )} - 1\end{matrix}} \qquad ( A )$$
x_{NMHC}: concentration of hydrocarbon component other than methane in sample gas
x_{THC (NMC-FID)}: concentration of methane measured by second analyzer
RF_{CH4 (THC-FID)}: response coefficient of methane in first analyzer
x_{THC (THC-FID)}: total hydrocarbon concentration measured by first analyzer
RF_{C2H6 (NMC-FID)}: response coefficient of ethane in second analyzer
PF_{C2H6 (NMC-FID)}: transmittance of ethane in second analyzer
K: moisture concentration correction coefficient

7. The gas analysis device described in claim 1,
in case that the second analyzer is calibrated by using methane and bypassing the non-methane cutter, wherein
the calculation section calculates the concentration of a hydrocarbon component other than methane in the sample as using the following expression (B).
$${x}_{NMHC} = \frac{\begin{matrix}K \cdot {x}_{THC ( NMC - FID )} \cdot {RF}_{{CH}_{4} ( THC - FID )} - \\ {x}_{THC ( THC - FID )} \cdot P {F}_{{CH}_{4} ( NMC - FID )}\end{matrix}}{\begin{matrix}{RF}_{{CH}_{4} ( THC - FID )} \cdot {RF}_{{C}_{2} {H}_{6} ( NMC - FID )} \cdot \\ {PF}_{{C}_{2} {H}_{6} ( NMC - FID )} - P {F}_{{CH}_{4} ( NMC - FID )}\end{matrix}} \qquad ( B )$$
x_{NMHC}: concentration of hydrocarbon component other than methane in sample gas
x_{THC (NMC-FID)}: concentration of methane measured by second analyzer
RF_{CH4 (THC-FID)}: response coefficient of methane in first analyzer
x_{THC (THC-FID)}: total hydrocarbon concentration measured by first analyzer
PF_{CH4 (NMC-FID)}: transmittance of methane in second analyzer
RF_{C2H6 (NMC-FID)}: response coefficient of ethane in second analyzer
K: moisture concentration correction coefficient

8. The gas analysis device described in claim 1,
in case that the second analyzer is calibrated by using propane and bypassing the non-methane cutter, wherein
the calculation section calculates the concentration of the hydrocarbon component other than methane in the sample gas using the following expression (C).
$${x}_{NMHC} = \frac{K \cdot {x}_{THC ( NMC - FID )} - {x}_{THC ( THC - FID )} \cdot P {F}_{{CH}_{4} ( NMC - FID )}}{{PF}_{{C}_{2} {H}_{6} ( NMC - FID )} - {PF}_{{CH}_{4} ( NMC - FID )}} \qquad ( C )$$
x_{NMHC}: concentration of hydrocarbon component other than methane in the sample gas
x_{THC (NMC-FID)}: concentration of methane measured by second analyzer
x_{THC (THC-FID)}: total hydrocarbon concentration measured by first analyzer
PF_{CH4 (NMC-FID)}: transmittance of methane in second analyzer
PF_{C2H6 (NMC-FID)}: transmittance of ethane in second analyzer
K: moisture concentration correction coefficient

9. The gas analysis device described in claim 1, further comprising
a third flow channel through which the sample gas flows,
a non-methane/non-ethane cutter that is arranged in the third flow channel and that removes the hydrocarbon component other than methane and ethane in the sample gas and
a third analyzer that is arranged downstream of the non-methane/non-ethane cutter in the third flow channel and that performs dry-measurement of the total methane/ethane concentration of methane and ethane in the sample gas, wherein
the calculation section calculates the concentration of the hydrocarbon component other than methane and ethane in the sample gas using the total hydrocarbon concentration obtained by the first analyzer and the total corrected concentration of methane and ethane, which is the moisture-corrected total concentration of methane and ethane, obtained by the third analyzer.

10. The gas analysis device described in claim 9, wherein
in case that the third analyzer is calibrated by passing ethane through the non-methane/non-ethane cutter, wherein
the calculation section calculates the concentration of the hydrocarbon component other than methane in the sample gas according to the following expression (D).
$${x}_{NMNEHC} = \frac{{K}^{′} \cdot {x}_{THC ( NMNEC - FID )} - {x}_{THC ( THC - FID )}}{{PF}_{{C}_{3} {H}_{8} ( NMNEC - FID )} - 1} \qquad ( D )$$
x_{NMNHC}: concentration of hydrocarbon component other than methane and ethane in sample gas
x_{THC (NMNEC-FID)}: total concentration of methane and ethane measured by third analyzer
x_{THC (THC-FID)}: total hydrocarbon concentration measured by first analyzer
PF_{C3H8 (NMNEC-FID)}: transmittance of propane in third analyzer
K′: moisture concentration correction coefficient

11. A gas analysis device comprising
a first flow channel through which a sample gas flows,
a first analyzer that is arranged in the first flow channel and that performs wet-measurement of a total hydrocarbon concentration in the sample gas,
a second flow channel through which the sample gas flows,
a non-methane/non-ethane cutter that is arranged in the second flow channel and that removes a hydrocarbon component other than methane and ethane in the sample gas,
a second analyzer that is arranged downstream of the non-methane/non-ethane cutter in the second flow channel and that performs dry-measurement of a total concentration of methane and ethane in the sample gas, and
a calculation section that calculates the concentration of the hydrocarbon component other than methane and ethane in the sample gas using the total hydrocarbon concentration obtained by the first analyzer and the total corrected concentration of methane and ethane, which is the moisture-corrected total concentration of methane and ethane, obtained by the second analyzer.

12. (canceled)

13. A gas analysis method comprising steps of
arranging a first analyzer that performs wet-measurement of a total hydrocarbon concentration in a sample gas in a first flow channel through which the sample gas flows,
arranging a hydrocarbon selective catalyst that removes a predetermined hydrocarbon component in the sample gas in a second flow channel through which the sample gas flows,
arranging a second analyzer that performs dry-measurement of the concentration of the hydrocarbon component in the sample gas downstream of the hydrocarbon selective catalyst in the second flow channel, and
calculating the concentration of the predetermined hydrocarbon component in the sample gas using the total hydrocarbon concentration obtained by the first analyzer and a corrected concentration of hydrocarbon component, which is a moisture-corrected concentration of the hydrocarbon component, obtained by the second analyzer.
